(19) United States
(12) Patent Application Publication
(10) Pub. No.: US 2017/0367882 A1
(43) Pub. Date: Dec. 28, 2017

(54) Dosage Delivery in Miniature Dispensing Pumps

(71) Applicant: Nigel KELLY, Rye Brook, NY (US)

(72) Inventor: Nigel KELLY, Rye Brook, NY (US)

(21) Appl. No.: 15/528,265

(22) PCT Filed: Dec. 31, 2015
(86) PCT No.: PCT/US2015/068362

§ 371 Date: May 19, 2017

Related U.S. Application Data
(60) Provisional application No. 62/081,678, filed on Nov. 19, 2014.

(51) Int. Cl. A61F 9/00; G01F 11/10

(52) U.S. Cl. CPC A61F 9/0026; G01F 11/10

(57) ABSTRACT

A meniscus compensating device for a low dosage fluid dispenser, the dispenser having a pump that is fluidly connectable between a pump outlet and a nozzle portion of the dispenser. The pump is operable to dispense a predetermined dose of the fluid through the pump outlet toward the nozzle portion. The meniscus compensating device has a fluid displacer, operable to displace a predetermined volume provided within the fluid column defined between the pump outlet and the nozzle mouth of the nozzle portion and the fluid displacer displaces the predetermined volume during dispensation of a releasing dose from the dispenser pump. The fluid displacer is at least partially released as or after the pump outlet is closed following the dose dispensation, thereby enabling the fluid column defined between the pump outlet and the nozzle mouth of the nozzle portion to occupy the previous displaced predetermined volume, thereby causing the meniscus formed after the dose dispensation at the terminal end of the fluid column at or near the nozzle mouth to come to rest at a substantively consistent predetermined position at or within the nozzle mouth.

This application claims the benefit of U.S. Provisional Patent Application No. 62/081,678, filed on Nov. 19, 2014.

### BACKGROUND OF THE INVENTION

This invention relates to miniature dispensing pumps, primarily used to deliver pharmaceuticals such as eye drops, or vaccines, or other liquids in small dose volumes.

It is common practice to prescribe eye drops for ophthalmic treatments. Consequently, it is desirable to develop apparatus and devices that may assist in the accurate application of ophthalmic fluids. Furthermore, it is preferred that the fluids are delivered in accurate small doses, such as approximately 5 to 50 micro-liters. Moreover, the ophthalmic fluids are preferably dispensed in small single droplets that avoid making noticeable impact on the eye.

To ensure that the fluids are delivered properly, a number of dispensers have been developed. However, most of these dispensers have shortcomings such as inaccurate dosage, frequent wastage and high-impact delivery. In addition, some of the existing dispensers have complicated structures that require precision injection molding and complex assembly, making the devices more costly to manufacture.

The present invention addresses the need for improved ophthalmic fluid delivery; a series of embodiments of this invention are illustrated in the accompanying drawings and are described in more detail herein below.

The use, operation and application of dispensing pumps are relevant, but in no way limited, to the devices disclosed in co-pending U.S. patent application Ser. No. 14/118,353, entitled “Fluid Dispenser”, filed on Nov. 18, 2013 as a National Stage application of PCT/US2012/038564 and claiming priority from U.S. Provisional Patent Application Ser. No. 61/487,349, filed on May. 18, 2011, and in co-pending U.S. patent application Ser. No. 14/235,212, entitled “Reservoir Module for Pump Dispenser”, filed on Jan. 27, 2013 as a National Stage application of PCT/US2012/048247 and claiming priority from U.S. Provisional Patent Application Ser. No. 61/512,377, filed on Jul. 27, 2011; these disclosures are incorporated herein by reference.

### SUMMARY OF THE INVENTION

According to a first aspect of the invention there is provided a meniscus compensating device for a low dosage fluid dispenser, the dispenser having a pump therein that is fluidly connectable between a pump outlet and a nozzle portion of the dispenser, and the pump being operable to dispense a predetermined dose of the fluid through the pump outlet toward the nozzle portion, the meniscus compensating device comprising:
a fluid displacer, operable to displace a predetermined volume provided within the fluid column defined between the pump outlet and the nozzle mouth of the nozzle portion;
wherein the fluid displacer displaces the predetermined volume during dispensation of a releasing dose from the dispenser pump; and
further wherein the fluid displacer is at least partially released as or after the pump outlet is closed following the dose dispensation, thereby enabling the fluid column defined between the pump outlet and the nozzle mouth of the nozzle portion to occupy the previously displaced predetermined volume, thereby causing the meniscus formed after the dose dispensation at the terminal end of the fluid column at or near the nozzle mouth to come to rest at a substantively consistent predetermined position at or within the nozzle mouth.

Typically, the fluid displacer displaces a predetermined volume equivalent to and counteracting the volume of the fluid that is driven toward the nozzle mouth as a result of the second tube folding location moving from its ‘open’ to its ‘closed’ positions immediately after the dispensed dose is released, thereby positioning the meniscus at or within the nozzle mouth.

Typically the fluid column is formed in a tube, preferably a resilient compressible tube or compliant tube, and the fluid displacer is operable to at least partially compress the fluid column defined between the pump outlet and the nozzle mouth of the nozzle portion. The fluid displacer is generally an anvil operable to be pressed against a segment of the tube between the pump outlet and the nozzle mouth.

The invention extends to a dispenser, having a meniscus compensating device.

According to a second aspect of the invention, there is provided a prime reduction device for a low dosage fluid dispenser, the dispenser having a pump therein that is fluidly connectable via a conduit between the pump inlet and the dispenser reservoir, the prime reduction device comprising a conduit volume reducing device, operable to be placed either at least partially within the conduit to occupy volume and thereby reduce the volume available for the fluid in the conduit, or to be applied externally and caused to exert pressure on the conduit to reduce the cross-section of at least a portion of the conduit and thereby reduce the volume available for the fluid within the conduit.

The conduit is typically a tube, generally a flexible, resilient compressible tube or compliant tube. The conduit volume reducing device is typically another tube of smaller bore which is inserted at least partially within the conduit tube.

Alternatively, the conduit volume reducing device may be one or more externally applied clips that are operable to compress the tube. The clips may be bridged together.

The invention extends to a dispenser, having a prime reduction device.

### BRIEF DESCRIPTION OF THE DRAWINGS

FIG. 1 shows a simplified cross-sectional cutaway view of a dispenser, incorporating a Z-pump, in a “rest” condition;

FIG. 2 shows an enlarged view of the nozzle portion of the Z-pump illustrated in FIG. 1;

FIG. 3 shows the dispenser in FIG. 1, with the Z-pump in its first stage of operation;

FIG. 4 shows the dispenser in FIG. 1, with the Z-pump in its second stage of operation;

FIG. 5 shows the dispenser in FIG. 1, with the Z-pump in its third stage of operation;

FIG. 6 shows the dispenser in FIG. 1, with the Z-pump in its fourth stage of operation;

FIG. 7 shows a simplified cross-sectional cutaway view of a first embodiment of a prime reduction device of a dispenser;

FIG. 8 shows a simplified cross-sectional cutaway view of a second embodiment of a prime reduction device of a dispenser;

FIG. 9 shows a simplified cross-sectional cutaway view of a third embodiment of a prime reduction device of a dispenser;

FIG. 10 shows an enlarged view of the prime reduction device of FIG. 9;

FIG. 11 shows a first sectional view of the prime reduction device of FIG. 9; and

FIG. 12 is a second sectional view of the prime reduction device of FIG. 9.

The illustrations are intended to provide a general understanding of the concepts described and the structure of various embodiments, and they are not intended to serve as a complete description of all the elements and features of methods and systems that might make use of the structures or concepts described herein. Many other embodiments will be apparent to those of skill in the art upon reviewing the description. Other embodiments may be utilized and derived therefrom, such that structural and logical substitutions and changes may be made without departing from the scope of this disclosure.

It should also be appreciated that the figures are merely representational, and are not necessarily drawn to scale, and certain proportions thereof may be exaggerated while others may be minimized. Accordingly, the specification and drawings, together with any examples, are to be regarded in an illustrative rather than a restrictive sense and the specific form and arrangement of the features shown and described are not to be understood or interpreted as limiting on the invention.

### DESCRIPTION OF EMBODIMENTS

In a first aspect of the present invention, the issue of meniscus compensation is addressed. The precise delivery of liquids from dispensers, such as those dispensers incorporating the Z-pump (as described in U.S. patent application Ser. No. 14/118,353, entitled “Fluid Dispenser”, detailed more completely above), depend for their accuracy and repeatability upon the liquid meniscus of the dose consistently being formed and coming to rest at a particular predetermined rest position in the nozzle portion after each operation of the pump. Preferably, this rest position is located at the mouth of an outlet nozzle or orifice, or alternatively it may be preferable in certain circumstances to have the meniscus come to rest at a predetermined position within the nozzle portion.

Some pumps, after liquid delivery, may intend to have the meniscus of the remaining liquid in the dispenser return to the same position after each operation of the pump, but in practice the valves employed in these pumps, such as check valves, generally cause the meniscus to retreat back from the mouth of the dispenser nozzle in an uncontrolled or random manner as the moving elements of the valve close onto its seat. It is especially important that the liquid meniscus comes to rest consistently at substantively the same rest position after each activation of a dose in low dose pumps, both to ensure precise dose-to-dose accuracy and also to ensure that the dispensed liquid neither spills from the nozzle nor is it pulled back into the pump, potentially drawing in contaminants that may be present on the outside of the dispenser. Therefore, it is desirable to restore the meniscus to a constant position flush with, or within, the nozzle mouth after each dose of dispensed liquid has been delivered.

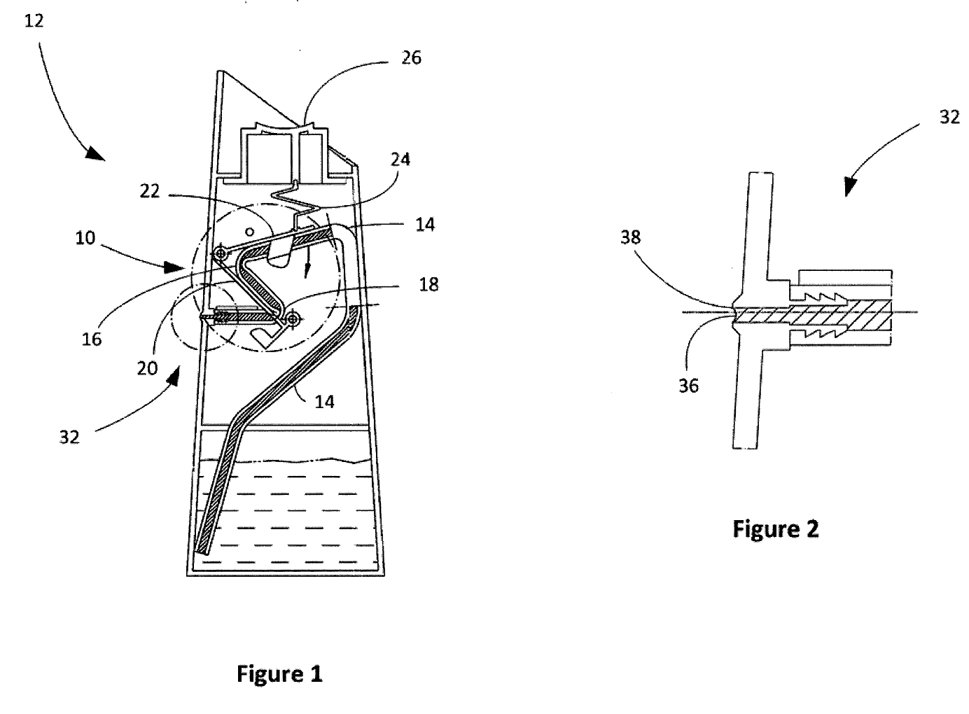

An embodiment of the present invention with regard to meniscus compensation can be understood with reference to FIGS. 1 through 6, showing the operational stages of a micro-dispensing Z-pump 10 within a dispenser 12. It will be appreciated that the incorporation of the Z-pump mechanism is not to be understood as a necessary or limiting requirement for the application of the present invention, and is described and illustrated here merely to disclose an example of a working manifestation of the present invention.

In FIG. 1, the Z-pump 10 mechanism is shown at rest within the dispenser 12. The Z-pump shown relies upon a dosage tube 14 being longitudinally foldable at a first tube folding location 16 (shown open) and a second tube folding location 18 (shown closed). The dosage tube is operable to be articulated between hingedly connected first and second hinge pieces (20 and 22 respectively) via activation of a spring member 24, connecting a push button 26 to the second hinge piece. Consequently, the first and second tube folding locations form the pump inlet and pump outlet, respectively.

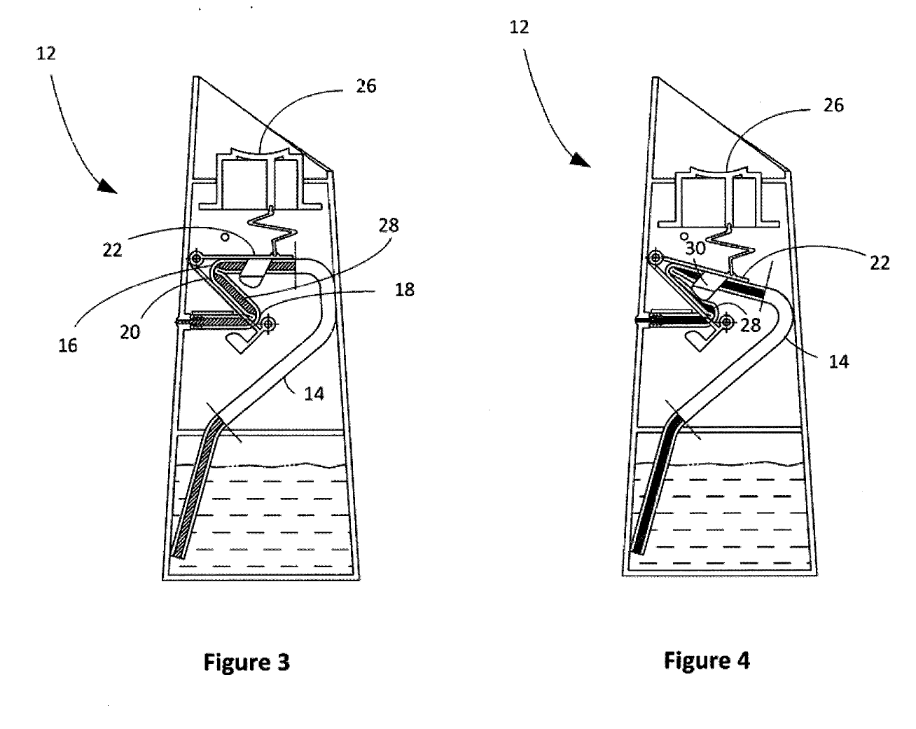

FIG. 3 shows the Z-pump as the push button 26 is partially activated in the first stage of operation; causing a predetermined dosage volume 28 of liquid to be entrapped between the first tube folding location 16 (shown closed) and the second tube folding location 18 (shown closed) as the first and second hinge pieces (20, 22) are rotated closer together. Consequently, the first and second tube folding locations form the pump inlet and pump outlet, respectively, and a dosing chamber is created for the dosage volume.

FIG. 4 shows the second stage of operation, where the push button 26 has been mostly activated. In this position, a primary anvil 30 carried on the second hinge piece 22 squeezes the dosage tube 14, thereby generating pressure in the dosage volume 28. FIG. 5 shows the third stage of operation, wherein the first and second hinge pieces (20, 22), in their compressed configuration, are able to rotate together; thereby enabling the opening at the second tube folding location 18 and releasing the pressurized dosage volume. The pump delivers the dose as it moves through the positions shown in FIGS. 4, and 5, during which the initially closed second tube folding location 18 begins to open up, and thereby allows the pressurized liquid to escape from the pump outlet toward the nozzle portion 32 and displace the fluid column line in the outlet section 33 of the dosage tube (downstream of the second tube folding location) and the nozzle portion. It should be appreciated that the volume of the dose delivered is determined by the dosage volume 28, which may partially or completely displace the fluid column line initially stored between the second tube folding location and the outlet of the nozzle portion.

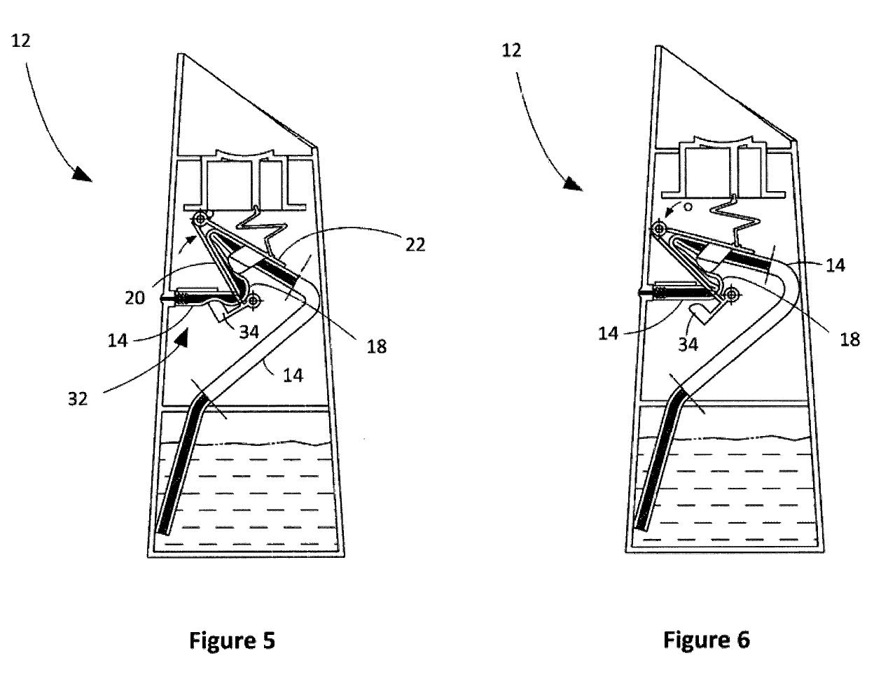

As is evident from the figures, the present invention incorporates a fluid displacer, described in this embodiment in the form of a secondary anvil 34. Without this secondary anvil, as the Z-pump 10 articulates after the aforementioned dosing stage of operation, and subsequently returning to the rest configuration shown in FIG. 1, the articulation of the second tube folding location 18 through the stages shown in FIGS. 5 and 6 has the effect of displacing the meniscus 36 slightly beyond the nozzle mouth 38 of the nozzle portion 32 (see FIG. 2 in particular). This displacement can be detrimental since it may cause the meniscus to break and liquid to drip from the nozzle mouth and onto the outside of the dispenser 12.

However, with the secondary anvil 34, carried on the first hinge piece 20, during the third stage of operation shown in FIG. 5, an outlet fluid displacer is applied in the form of the secondary anvil impinging against the dosage tube 14 at or near the nozzle portion 32 as dosing begins. As the pump articulates and rotates back, after dosing, the pump outlet at the second tube folding location 18 closes (shown in FIG. 6), and in so doing this causes the meniscus 36 at the head of the fluid column line to translate slightly outwardly toward the nozzle mouth 38. At the same time as this happens, the secondary anvil that was pressing against the dosage tube and displacing fluid retracts away from the tube—thereby allowing the tube at that point to be restored to its fully round cross-sectional shape and thereby to increase the volume of the outlet section of the tubing adjacent the nozzle portion.

The operation of the secondary anvil 34 is consequently designed to increase the volume in the outlet section of the dosage tube 14 by an amount that is exactly equal to the amount of liquid volume that is being driven outwardly toward the nozzle portion 32 as the fold at the second tube folding location 18 closes (from positions shown in FIG. 5 to FIG. 6). The effect of the secondary anvil essentially counteracts the tendency for the fold in the tube at the second tube folding location to ‘push’ the meniscus 36 outwardly toward the nozzle mouth 38, by providing an equal and opposite ‘pull’ of the meniscus inwardly from the nozzle mouth. The balancing of these two systems (the ‘push’ caused by the folding action of the tube, and the ‘pull’ caused by the secondary anvil moving away from the tube) ensures that the meniscus does not move along the tube in either direction during the final (fourth) stage of pump operation and as it returns to the rest position.

Ideally, the compensating device will restore the meniscus so that the outer extent of the meniscus will be level with the mouth of the nozzle. It will be appreciated that the meniscus formed in accordance with the present invention will be concave, facing inwardly toward the nozzle mouth, in all cases except when the meniscus is pushed past the nozzle mouth; at which point it will go convex until it breaks. Since it is preferable for the meniscus to remain at or within the nozzle mouth, it will therefore generally be concave in form. In certain circumstances, however, for example where cross contamination of the dosing applied to both eyes is being avoided, the meniscus formation may be designed to rest at a constant position a short distance inside the nozzle after each pump operation.

In a second aspect of the invention, pump priming is addressed. Dispenser pumps which rely on flexible tubing to carry the dispensed liquid into the pump, such as the Z-pump, must first be ‘primed’ before any liquid emerges from the pump nozzle. With low dose, or micro-dosing pumps, priming can require more strokes of the pump than might be desirable to the user, or the user may assume the dispenser is not working or empty. Means for reducing the number of strokes required to prime such pumps is advantageous.

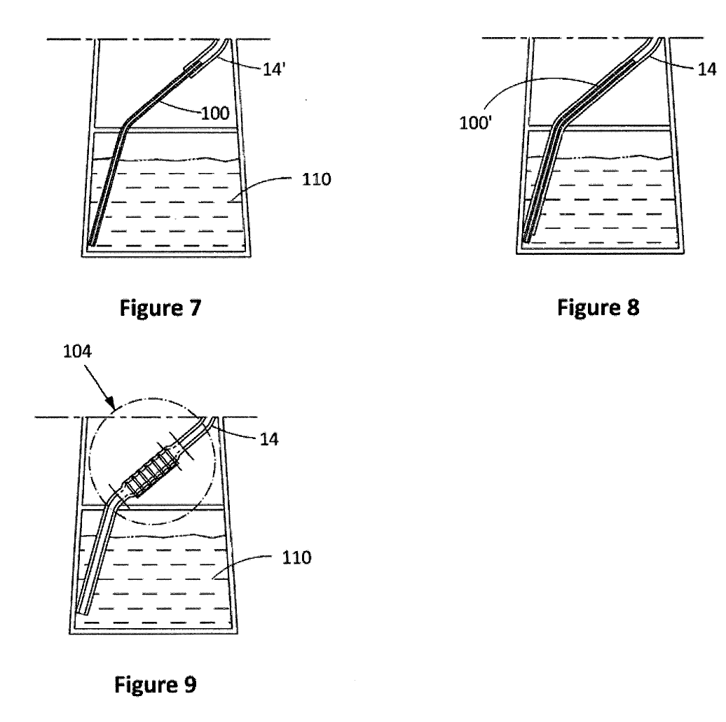

FIG. 7 shows a first embodiment for reducing the number of strokes to prime the pump (not shown). This involves the use of more than one size of tube 14′ along the fluid path within the sections of tube that are not involved in the active pump mechanism; such as tubing defined between the reservoir and the upstream folds in the tube of a Z-pump. In this manner, tubing 100 is provided of smaller bore size than the tubing employed within the active sections of the Z-pump; the smaller bore sizes of tubing require less liquid volume to prime the pump along the tube length, resulting in fewer strokes being required to prime the system as a whole.

FIG. 8 shows another embodiment for reducing the number of strokes to prime the pump. A smaller diameter tube 100′ is inserted inside a large tube 14, such that the small tube 100′ occupies volume within the large tube thereby reducing strokes required for priming.

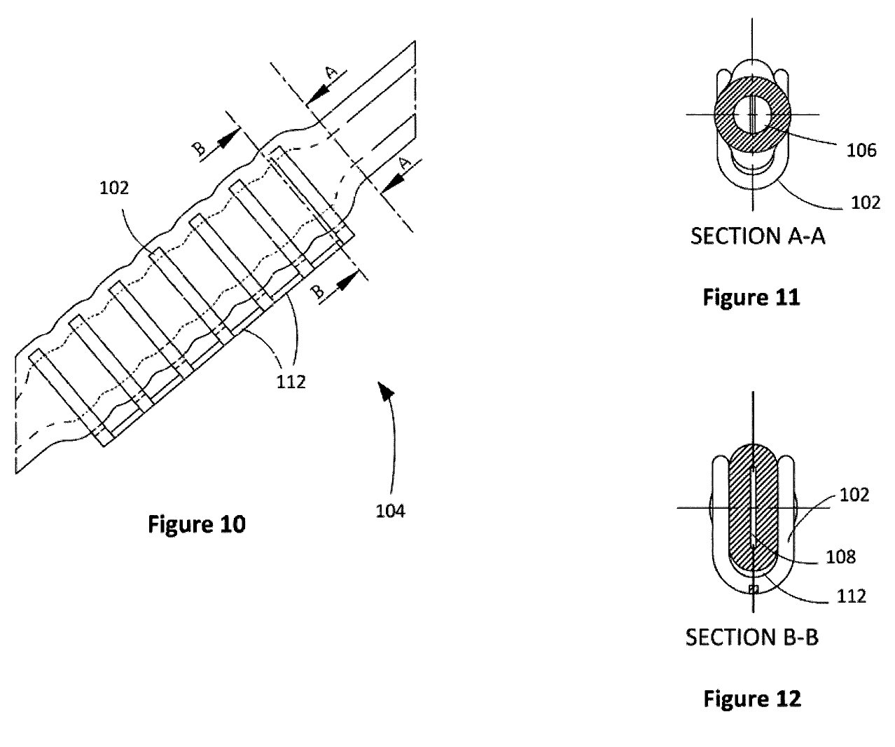

FIGS. 9 through 12 show yet another embodiment of a prime-reduction device, consisting of the installation of a horseshoe-shaped clip 102, or a series of horseshoe-shaped clips 104, that partially squeeze the tube 14 between the reservoir and the upstream folds; thereby reducing the internal volume within the tubing. FIG. 11 shows a cross section (A-A from FIG. 10) of the tube in its relaxed condition and defining an open, rounded cross-section 106. FIG. 12 shows a cross section (B-B from FIG. 10) of the tube in its squeezed condition and defining a flattened reduced section 108. Consequently, the amount of liquid 110 required to fill the tubing between the reservoir and the pump mechanism, in order to complete the priming of the pump, is significantly reduced. The clips can take the form of a single horseshoe-shaped clip 102, or multiple clips can be connected in a line via bridges 112. To permit easier articulation of the flexible tubing they surround, the bridges may themselves be made from a narrow section material which bends freely and allows the assembled line of clips to follow the articulation of the tube without restriction. In the case of the Z-pump, certain sections of its tubing need to bend or to be folded or crushed in order to allow the pump to operate properly. These particular sections clearly would not be suitable locations for the clips. Clips would instead be attached to the tubing at positions where the tubing is not required to fold or be squeezed within the confines of the active pump mechanism; primarily nearer the reservoir.

1. A meniscus compensating device for a low dosage fluid dispenser, the dispenser having a pump therein that is fluidly connectable between a pump outlet and a nozzle portion of the dispenser, and the pump being operable to dispense a predetermined dose of the fluid through the pump outlet toward the nozzle portion, the meniscus compensating device comprising:
a fluid displacer, operable to displace a predetermined volume provided within a fluid column defined between the pump outlet and a nozzle mouth of the nozzle portion;
wherein the fluid displacer displaces the predete ned volume during dispensation of a releasing dose from the dispenser pump; and
further wherein the fluid displacer is at least partially released as or after the pump outlet is closed following the dose dispensation, thereby enabling the fluid column defined between the pump outlet and the nozzle mouth of the nozzle portion to occupy the previously displaced predetermined volume, thereby causing a meniscus formed after the dose dispensation at a terminal end of the fluid column at or near the nozzle mouth to come to rest at a substantively consistent predetermined position at or within the nozzle mouth.

2. The meniscus compensating device of claim 1, wherein the fluid displacer displaces a predetermined volume equivalent to and counteracting the volume of the fluid that is driven toward the nozzle mouth as a result of a second tube folding location moving from an open to a closed position immediately after the dispensed dose is released, thereby positioning the meniscus at or within the nozzle mouth.

3. The meniscus compensating device of claim 1, wherein the fluid column is formed in a tube, preferably a resilient compressible tube or compliant tube, and the fluid displacer is operable to at least partially compress the fluid column defined between the pump outlet and the nozzle mouth of the nozzle portion.

4. The meniscus compensating device of claim 1, wherein the fluid column is formed in a tube, preferably a resilient compressible tube or compliant tube, and the fluid displacer is operable to at least partially compress the fluid column defined between the pump outlet and the nozzle mouth of the nozzle portion, and wherein the tube is a resilient compressible tube or compliant tube and the fluid displacer is operable to at least partially compress the fluid column defined between the pump outlet and the nozzle mouth of the nozzle portion.

5. The meniscus compensating device of claim 1, wherein the fluid displacer is an anvil operable to be pressed against a segment of a tube between the pump outlet and the nozzle mouth.

6. A prime reduction device for a low dosage fluid dispenser, the dispenser having a pump therein that is fluidly connectable via a conduit between a pump inlet and a dispenser reservoir, the prime reduction device comprising a conduit volume reducing device, operable to be placed either at least partially within the conduit to occupy volume and thereby reduce the volume available for the fluid in the conduit, or to be applied externally and caused to exert pressure on the conduit to reduce the cross-section of at least a portion of the conduit and thereby reduce the volume available for the fluid within the conduit.

7. The prime reduction device of claim 6, wherein the conduit is a tube.

8. The prime reduction device of claim 6, wherein the conduit is a flexible, resilient compressible tube or compliant tube.

9. The prime reduction device of claim 6, wherein the conduit is a tube and the conduit volume reducing device is a tube of smaller bore which is inserted at least partially within the conduit tube.

10. The prime reduction device of claim 6, wherein the conduit is a tube and the conduit volume reducing device is one or more externally applied clips that are operable to compress the tube.

11. The prime reduction device of claim 6, wherein the conduit is a tube and the conduit volume reducing device is formed from at least two externally applied clips that are operable to compress the tube and the clips are bridged together.

12. A low dosage fluid dispenser, comprising:
a pump, the pump being fluidly connectable between a pump outlet and a nozzle portion of the dispenser, and the pump being operable to dispense a predetermined dose of the fluid through the pump outlet toward the nozzle portion;
a meniscus compensating device, having a fluid displacer, operable to displace a predetermined volume provided within a fluid column defined between the pump outlet and a nozzle mouth of the nozzle portion;
wherein the fluid displacer displaces the predetermined volume during dispensation of a releasing dose from the dispenser pump; and
further wherein the fluid displacer is at least partially released as or after the pump outlet is closed following the dose dispensation, thereby enabling the fluid column defined between the pump outlet and the nozzle mouth of the nozzle portion to occupy the previously displaced predetermined volume, thereby causing a meniscus formed after the dose dispensation at a terminal end of the fluid column at or near the nozzle mouth to come to rest at a substantively consistent predetermined position at or within the nozzle mouth.

13. A low dosage fluid dispenser, comprising:
a pump, the pump being fluidly connectable via a conduit between a pump inlet and a dispenser reservoir,
a prime reduction device, having a conduit volume reducing device, operable to be placed either at least partially within the conduit to occupy volume and thereby reduce the volume available for the fluid in the conduit, or to be applied externally and caused to exert pressure on the conduit to reduce the cross-section of at least a portion of the conduit and thereby reduce the volume available for the fluid within the conduit.
